(19) United States
(12) Patent Application Publication
(10) Pub. No.: US 2024/0331869 A1
(43) Pub. Date: Oct. 3, 2024

(54) Fever Prediction

(71) Applicant: 37 CLINICAL LIMITED, Cambridge (GB)

(72) Inventors: Harald Braun, Cambridge (GB); Claire Hooper, Cambridge (GB)

(21) Appl. No.: 18/577,349

(22) PCT Filed: Jul. 8, 2022
(86) PCT No.: PCT/GB2022/051778

§ 371 Date: Jan. 8, 2024

(30) Foreign Application Priority Data
Jul. 8, 2021 (GB) 2109899.1
Jul. 8, 2021 (GB) 2109900.7

(51) Int. Cl. G16H 50/30; G16H 30/40; G16H 50/20

(52) U.S. Cl. CPC G16H 50/30; G16H 30/40; G16H 50/20

(57) ABSTRACT

A computer-implemented method of predicting fever comprising: receiving a data sequence representing a physiological parameter of a user over a first period: transforming the data sequence so as to form a scalogram representing the physiological parameter of the user over a second period: analysing the scalogram at a neural network adapted to perform image classification so as to identify one or more fever precursors in the scalogram characteristic of the onset of fever; and in response to identifying at least one of the one or more fever precursors in the scalogram, providing a prediction of the onset of fever in the user.

### BACKGROUND OF THE INVENTION

This present disclosure relates to methods and systems for predicting the onset of fever in humans and animals.

Fever can present in humans and animals for a variety of reasons, including as a result of immune responses to viral and bacterial pathogens, Chronic Obstructive Pulmonary Disease (COPD), neutropenia, and cystic fibrosis. The detection of fevers is typically performed manually through the use of a thermometer. For example, patients in hospital at risk of developing a fever may have their temperature regularly checked. In the home environment, a person will typically check their temperature only should they have reason to believe they have a fever—e.g. due to feeling unwell and/or hot or cold.

Through the use of body worn temperature sensors, it is possible to continuously record the temperature of a person. By monitoring the recorded temperature it is possible to identify the onset of fever in a person by detecting when the body temperature of the person exceeds a predetermined level (this can vary depending on the location of the sensor, but for core body temperature is typically a temperature greater than 38 deg C). This approach can ensure that fevers are identified as early as possible.

US Patent Publication No. 2021/0100454 describes a machine learning system for analysing data from a continuous temperature monitor so as to predict variations in temperature one hour into the future. By examining the predicted temperature data for fever events, the onset of fever may be quickly detected.

### SUMMARY OF THE INVENTION

There is provided a computer-implemented method of predicting fever comprising:
receiving a data sequence representing a physiological parameter of a user over a first period;
transforming the data sequence so as to form a scalogram representing the physiological parameter of the user over a second period;
analysing the scalogram at a neural network adapted to perform image classification so as to identify one or more fever precursors in the scalogram characteristic of the onset of fever; and
in response to identifying at least one of the one or more fever precursors in the scalogram, providing a prediction of the onset of fever in the user.

There is provided a data processing system for predicting fever comprising:
an input unit configured to receive a data sequence representing a physiological parameter of a user over a first period;
a transformation unit configured to transform the data sequence so as to form a scalogram representing the physiological parameter of the user over a second period;
a neural network adapted to perform image classification and arranged to analyse the scalogram so as to identify one or more fever precursors in the scalogram characteristic of the onset of fever; and
an output unit configured to, in response to identifying at least one of the one or more fever precursors in the scalogram, provide a prediction of the onset of fever in the user.

There is provided a computer-implemented method of training a neural network to predict fever, the method comprising:
for each of a plurality of datasets each comprising temperature data of a user:
   receiving a dataset comprising temperature data of a user;
   analysing the temperature data to identify an onset of a fever event;
   in response to identifying a fever event in the temperature data, forming a training data sequence comprising temperature data of the user over a predefined period prior to the onset of the fever event and classifying the training data sequence as relating to a fever event;
and
   training a neural network to identify precursors characteristic of the onset of fever using the plurality of training data sequences.

There is provided a computer-implemented method of training a neural network to predict fever, the method comprising:
receiving time series data representing a physiological parameter of one or more users;
using the time series data:
   forming a first set of one or more training data sequences associated with a fever event, the training data sequences each representing the physiological parameter over a predefined period of time prior to the onset of the fever event; and
   forming a second set of one or more training data sequences not associated with a fever event, the training data sequences representing the physiological parameter over the predefined period of time; and
training a neural network to identify precursors characteristic of the onset of fever using the first and second sets of training data sequences.

Further features of the methods are as set out in the claims.

### BRIEF DESCRIPTION OF THE DRAWINGS

The present invention is described by way of example with reference to the accompanying drawings. In the drawings:

FIG. 1 illustrates a data processing system for predicting fever in accordance with the principles described herein.

FIG. 2 shows a time series comprising three days of temperature data prior to a fever event.

FIG. 3 shows a temperature time series captured by a wearable sensor which includes a data drop-out and a fever event.

FIG. 4 illustrates the concept of predicting a fever by identifying fever precursors in a temperature time series.

FIG. 5 is an example of a scalogram.

FIG. 6 illustrates a set of time series being converted into scalograms using a continuous wave transform.

FIG. 7 shows a set of temperature bands for use in a multi-threshold approach to identifying artefacts and/or fever events in a time series.

FIG. 8 illustrates a mild fever event.

FIG. 9 illustrates a set of shower artefacts.

FIG. 10 is a flowchart illustrating the prediction of fever from continuously acquired body temperature data according to the principles described herein.

FIG. 11 is a flowchart illustrating training a neural network for predicting the onset of fever as described herein.

FIG. 12 illustrates a fever precursor along with mild and high fever events.

### DETAILED DESCRIPTION OF THE INVENTION

The following description is presented by way of example to enable a person skilled in the art to make and use the invention. The present invention is not limited to the embodiments described herein and various modifications to the disclosed embodiments will be apparent to those skilled in the art.

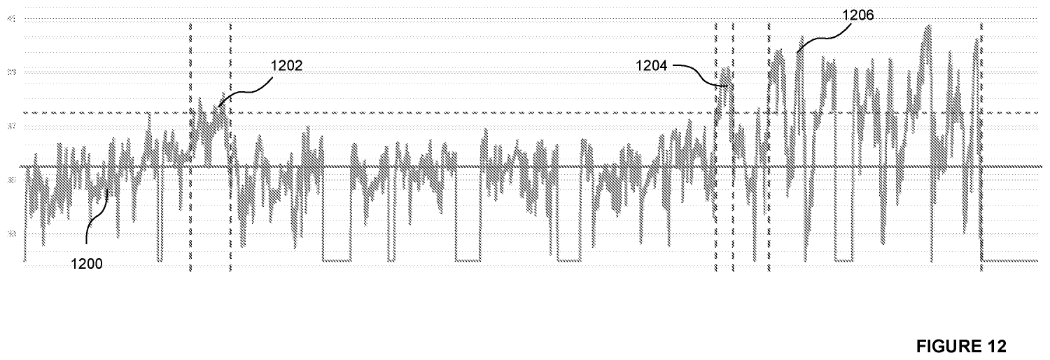

It has been established by the inventors that fevers can be preceded by characteristic pre-fever perturbations or precursors in physiological data captured from a person. Examples of suitable physiological data include temperature, movement, ECG, heart rate, heart rate variability, and respiratory data. These perturbations may be specific to the particular pathogen or disease causing the fever. The present disclosure provides systems and methods for detecting the possible onset of fever in the future (e.g. up to three days ahead) by identifying such characteristic precursors in advance of the fever itself. FIG. 12 shows an example of a fever precursor 1202 observed in a temperature time series 1200 of a user around two days prior to the onset of a mild fever 1204 which is subsequently followed by a high fever 1206 from which the user does not recover. Examples will be described herein with respect to temperature and movement data but it will be understood that the same principles of forming a scalogram from a time series of sensor data for processing at a neural network adapted to perform image classification can be applied to ECG, heart rate, heart rate variability, and respiratory data.

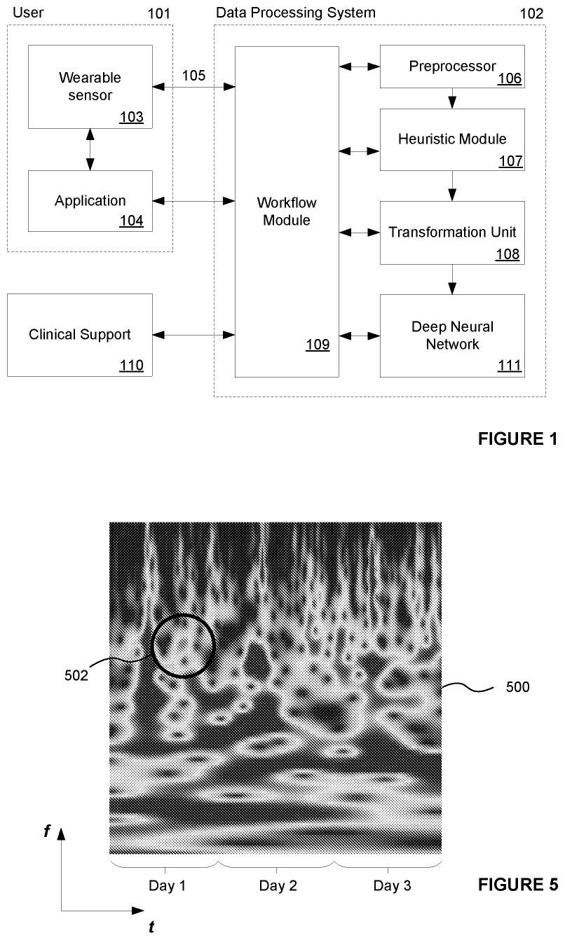

FIG. 1 illustrates a data processing system 102 for predicting fever in accordance with the principles described herein. The operation of the data processing system on temperature data is described herein by way of example but it shall be understood that the same principles may be applied to movement data captured from a user so as to identify fever precursors in the movement data (e.g. the amplitude of movement of a user over time). A user 101 (which may be a human or animal) has a suitable physiological parameter (e.g. their temperature) monitored by a wearable sensor 103. For example, the wearable sensor could be a temperature and/or movement sensor worn on the skin (e.g. on the chest under the armpit) or any other kind of sensor (e.g. an indwelling sensor or implant) suitable for measuring the temperature and/or capturing movement of the user. The wearable sensor 103 is a continuous monitor in that it is configured to periodically measure the temperature and/or movement of the user (e.g. every 30 seconds up to every few minutes).

In order to enable the detection of fever precursors, it is advantageous for the user's physiological parameters (e.g. temperature and/or movement) to be measured at least every ten minutes, and better performance can be achieved by measuring more frequently—e.g. at least every 5,4,3,2.1 minutes. It is envisaged that whilst the wearable sensor is a continuous monitor, it may not be permanently worn, and could be removed by the user (e.g. for bathing, while exercising, during the day, etc.). The wearable sensor may be configured to capture data only when it is in contact with the user (e.g. by means of a capacitive sensor as is known in the art; when the measured body temperature is within a predefined range which is suggestive that it is being worn—e.g. within a few degrees of 37 deg C; and/or when the measured movement is indicative that the sensor is being worn—e.g, movement detected by the sensor has exceeded a predefined threshold a predefined number of times within a predefined window of time).

The wearable sensor may comprise one or more temperature sensors. As is known in the art, the wearable sensor may include one or more sensors configured to correct a body temperature measurement. For example, a skin worn sensor may include a first sensor arranged to measure the temperature of the skin of the user and a second sensor arranged to measure environmental temperature. The environmental temperature may be used to correct (e.g. using a linear or higher order correction based on the difference between the skin and environmental temperatures) the body temperature measurements according to any suitable technique such that the temperature measurements provided by the sensor to the data processing system are the corrected temperature measurements. Such sensors may be referred to as heat flow temperature sensors.

The wearable sensor may comprise one or more movement sensors—for example, accelerometers. A movement sensor may provide any suitable movement data, such as vector data representing the size and direction of movement and/or amplitude data representing the size (e.g. amplitude of the movement vector) of movements captured by the sensor. In general, one or more wearable sensors may be provided—e.g. a temperature sensor and a movement sensor, which could be worn at different points on the body.

The wearable sensor is configured to provide the data it captures to the data processing system for analysis (labelled server 102 in the figure). The wearable sensor may provide information with the data sufficient to enable the data processing system to determine when each sensor value was measured—for example, a timestamp for each sensor value, or a starting time and a measurement frequency so as to enable the measurement time of each sensor value to be determined. The wearable sensor may be configured to communicate to the server by means of a network 105. For example, the wearable sensor may be operable to communicate over a local network (e.g. a WiFi, Bluetooth, 5G, or ZigBee network). The local network may be connected to the internet over which the server is accessible. The wearable sensor may be configured to transmit its sensor data by any suitable protocol (e.g. a secure connection over a web services protocol such as SOAP or REST). The wearable sensor may be configured to compress and/or encrypt its sensor data prior to transmission to the server.

In some examples, the wearable sensor may communicate over network 105 by means of a smartphone or other computer running an application 104. In some examples, the wearable sensor is a low power Bluetooth device configured to pass its data to a smartphone with which the sensor is paired. The smartphone may be connected to a local WiFi network accessible to the internet (e.g. via a router). A suitable application 104 running at the smartphone may be configured to transmit the data to the server 102. The wearable sensor may be configured to transmit its captured data in real-time (e.g. on capturing each measurement), or periodically as a batch of captured data (e.g. according to a predefined schedule, such as every 30 mins). The wearable sensor may comprise a memory for storing measurements. This enables the wearable sensor to continue to record data even if a local network or intermediate device (e.g. a smartphone running application 104) is not available.

The data processing system 102 comprises a workflow module 109 which is configured to control the analysis performed at the various units of the data processing system and communicate with the wearable sensor 103 and/or application 104 (e.g. so as to receive data captured by the sensor and/or other data, such as configuration data or other physiological information relating to the user). The workflow module may comprise one or more of software, firmware and hardware running at the data processing system.

A preprocessor 106 may be provided at the data processing system 102 and/or the wearable sensor 103 and/or the application 104. The preprocessor may perform data filtering, e.g. to remove noise in the data, as is known in the art. For example, the preprocessor may discard or mark as not for use data which does not appear to reflect true physiological body temperature. For example, temperature data which is indicative of the sensor having lost contact with the body of the user (e.g. any measurements below 31 degc), or environmental conditions (e.g. a hot shower) causing the temperature measurements to be an unreliable representation of body temperature. The preprocessor may be configured discard all temperature data which is outside a predefined range (e.g. 32-42 deg C. for a skin worn sensor). The preprocessor may be configured discard all temperature data which exhibits a rate of change greater than a predefined rate (e.g. greater than 0.25 degC, 0.3 degC, 0.4 degC, 0.5 degC per minute). The sensor 103 may be configured to capture temperature measurements at a predefined rate—for example, one measurement every 45 seconds.

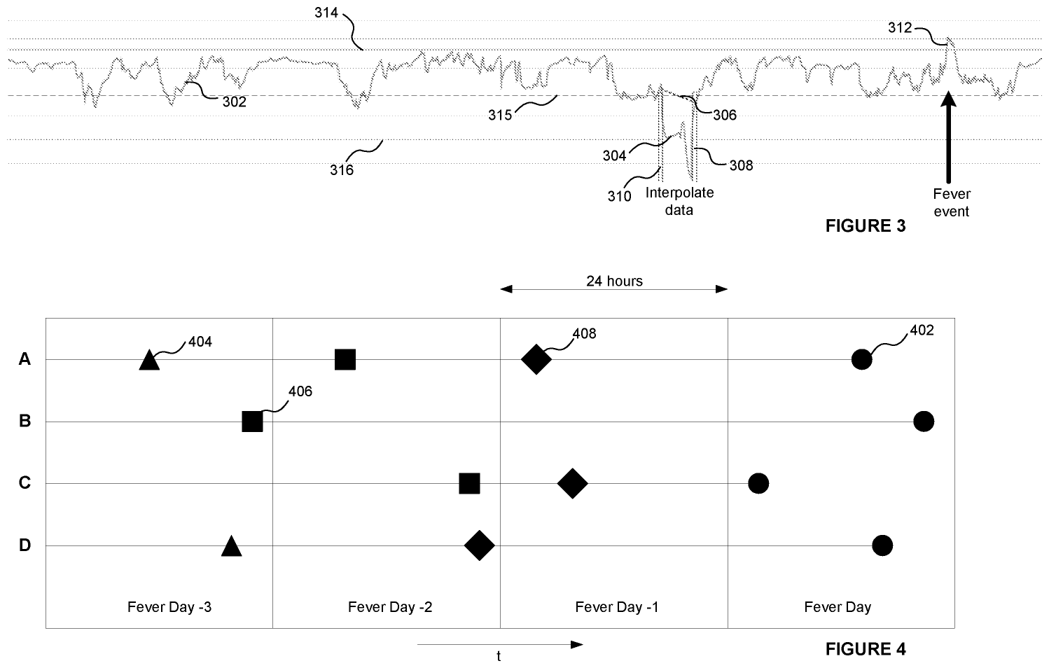

In respect of a given period of time, the data received at the preprocessor may be missing sensor measurements, for example due to measurements not being captured at the sensor (e.g. when the user removes the sensor) and/or due to filtering of the data at the preprocessor so as to remove temperature samples. In order to avoid gaps in the temperature data and to ensure that a complete set of temperature values exist for each given period of time, the preprocessor may be configured to interpolate missing temperature values or replace the missing values with suitable padding data (e.g. zeros). For example, FIG. 3 shows a temperature time series 302 captured by a wearable sensor in which a drop-out in the data occurs at 304—e.g. due to the user removing the sensor. By way of illustration the time series 302 in FIG. 3 includes a fever event 312.

An artefact 304 in the temperature measurements shown in FIG. 3 may be identified and/or removed by the filtering algorithms operating at the preprocessor and/or sensor itself. Since the temperature measurements 304 do not reflect true physiological body temperature, the preprocessor may be configured to replace the temperature measurements 304 with interpolated data 306. For example, the preprocessor could be configured to perform curve fitting to the temperature measurements in time periods either side 308, 310 of the drop-out event—e.g. the preprocessor may perform linear interpolation (as shown) or any other kind (e.g. non-linear) interpolation between those determined measurements so as to generate replacement temperature measurements 306. In some examples, the number of samples of the temperature data either side of the drop-out event used in the interpolation can depend on the width of the drop-out event. For example, the width of the time periods 308 and 310 could be 5%, 10%, or 15% of the width of the drop-out event 304. A weighted average algorithm may be used to perform the interpolation using the data for each of the time periods 308 and 310. A weighted average algorithm may use a window having a predefined width—e.g. 10, 15, 20, 25, or 30 samples wide.

The preprocessor and/or sensor may discard or mark as not for use data which does not appear to represent intrinsic movement of the user—i.e. movement due to extrinsic forces, such as being in a vehicle—and/or data which appears to represent movement due to exercise of the user. For example, movement data representing velocities and/or accelerations that exceed a predefined threshold may be discarded or marked as not for use. In order to avoid gaps in the movement data and to ensure that a complete set of movement values exist for each given period of time, the preprocessor and/or sensor may be configured to interpolate missing movement values or replace the missing values with suitable padding data (e.g. zeros). The sensor and/or preprocessor may be configured to perform filtering of the movement data so as to remove artefacts and sudden changes in movement. For example, the sensor and/or preprocessor may be configured to average movement values so as to generate a set of movement values for the neural network 111. In some examples, the sensor may be configured to capture each movement value as a value representative of an average (e.g. mean) of movement over some time period (e.g. at least one second, at least two seconds, or at least five seconds).

The workflow module 109 may be further configured to receive information provided by the user 101 and/or clinical support 110 (e.g. hospital staff caring for a patient). For example, the user may provide information (e.g. by means of application 104 running at their smartphone) to indicate periods of time when data is to be disregarded—e.g. because the user is having a bath/shower, has entered a sauna, has removed the sensor. In some examples, such information may be used by the workflow module to indicate to the preprocessor 106 and/or heuristic module 107 periods of time in which the sensor data is to be discarded. In response, the preprocessor 106 and/or heuristic module 107 may discard the associated data. In some examples, the sensor 130 and/or application 104 and/or workflow module 109 may be configured to discard the temperature data such that it is not provided to the preprocessor and/or heuristic module.

In the case of temperature sensors which are not indwelling so as to directly measure core body temperature, one or more of the sensor 103, preprocessor 106 and workflow module 109 may be configured to correct the captured temperature measurements so as to form an estimate of the core body temperature of the user. For example, through calibration of the temperature sensor based on its structure and its location on the body, a suitable temperature correction may be identified which can be added to all captured temperature values—e.g. for a skin temperature sensor worn on the chest under the arm, a 1.4 degC correction may be added to the temperature values measured at the skin by the sensor.

Note that a data processing system configured in accordance with the principles described herein need not operate on temperature data which is calibrated so as to reflect a true measure of core body temperature. For example, since skin temperature sensors typically measure temperature values which are lower than true core body temperature, the data processing system may be configured to operate according to the absolute level of temperature values provided by the sensor. E.g. for a skin temperature sensor worn on the chest under the arm which typically measures 1.4 degC below, the data processing system could be configured to operate at that level such that fevers are considered to occur when the measured temperature exceeds 36.7 degC (see Table 1 below).

In some examples, the sensor 103 and/or application 104 may comprise some of all of the data processing system 102 such that the data processing described herein is performed at the sensor and/or application.

### Heuristics

Optional heuristic module 107 is configured to identify artefacts and/or fevers in the measurements captured by wearable sensor 103. In some examples, preprocessor 106 may not be present and the heuristic module may receive data for processing according to the principles described below (e.g. in examples where the sensor 103 and/or application 104 perform filtering of the measurements and/or discarding of data values). The heuristic module preferably operates on a complete set of temperature values for the period concerned where any discarded or unavailable temperature measurements have been replaced with interpolated temperature values or padding data.

The heuristic module may be configured to apply an algorithm based on a multi-level threshold approach which identifies artefacts and/or fevers in the temperature data by dividing a temperature range into discrete bands and determining the time a user's body temperature spends in each band. Artefacts are temperature events in the data capture by the wearable sensor which do reflect the true underlying physiological temperature of the user due to external sources of heat (such as showers and baths) and excess heat is generated by the user as a result of exercise (which can be a particular issue for skin worn sensors). The identification of artefacts enables such events to be removed from the temperature data. Fever events are periods of increased temperature which appear to represent real increases in physiological temperature not as a result of exercise or external sources of heat. Fever events can be mild, moderate or severe and are typically clinically defined as follows:

TABLE 1

| Fever grade | Temperature (deg C.) |
|---|---|
| Mild/low | 38.1-39 |
| Moderate | 39.1-40 |
| High | 40.1-41.1 |
| Hyperpyrexia | >41.1 |

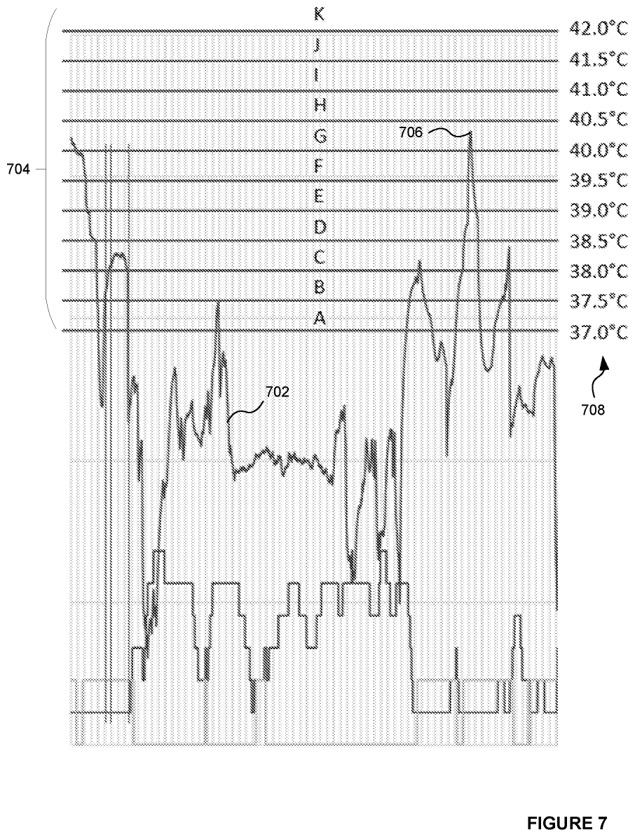

FIG. 7 shows a set of temperature bands 704 with respect to a temperature time series 702 captured by sensor 103. In this example, the temperature bands comprise 11 bands over the temperature range 708 from 37 degC to 42.5 degC, each band having a width of 0.5 degC. A larger or smaller number of bands may be used, each band being wider or narrower (e.g. in some examples the bands can be 0.7, 0.6, 0.3, 0.2, 0.1 degC in width). In some examples, different temperature bands may have different widths. In some examples, the temperature bands may cover temperatures below 37 degC.

The heuristic module 107 is configured to process the temperature data it receives (e.g. from the preprocessor 106) so as to identify the time the signal spends in each band. In this manner, the heuristic module can characterise peaks 706 in the received temperature time series as a sequence of discrete bands and the time the user spends in each band. This facilitates the use of rules that heuristically describe either physiological temperature events (e.g. fever, exercise) or extrinsic temperature events (e.g. hot showers).

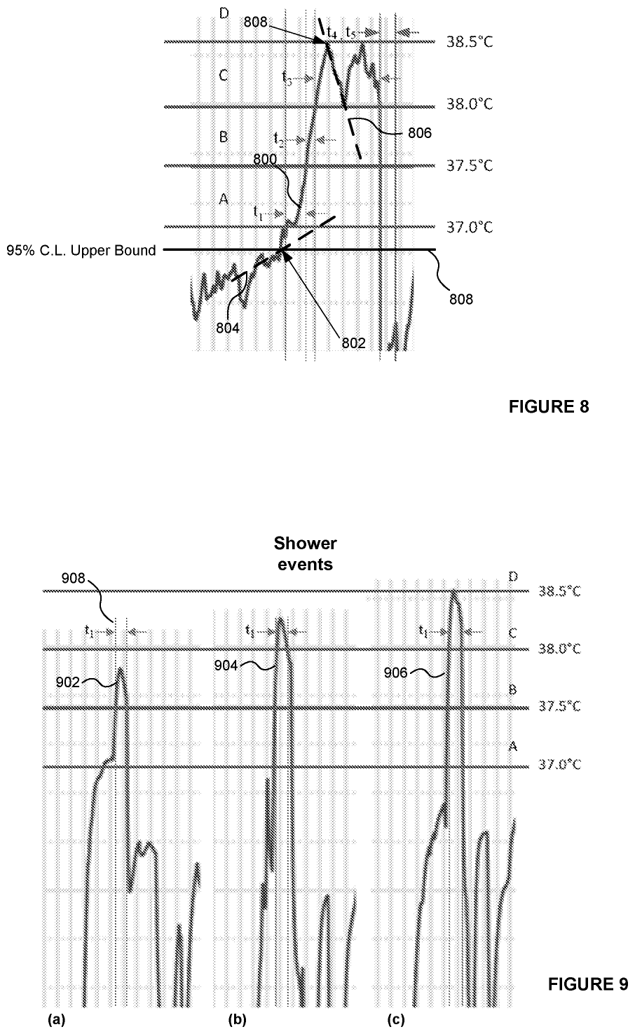

FIG. 8 shows an example of a mild fever event 800 with rapid onset from 37 degC to 38.5 degC in less than 90 minutes from a baseline at around 36.5 degC. The temperature data shown in the Figure is characterised by the heuristic module as spending a time interval t₁=55 minutes in band A as the temperature rises, a time interval t₂=22 minutes in band B as the temperature continues to rise, and a time interval t₃=165 minutes in band C where the temperature reaches a maximum indicative of a mild fever (see table 1), before a sharp drop through band B and A for a short time intervals t₄+t₅=15 minutes, which are shown together for clarity. Thus the temperature event 800 can be characterised as a sequence of bands along with the time at each band (a characteristic band sequence). For example: A (28 minutes), B (22 minutes), C (165 minutes), B (8 minutes), A (7 minutes). It is to be noted that the heuristic module need not explicitly form such a representation.

The heuristic module may be configured to identify an upper bound of an average baseline temperature for a user to a predefined confidence level (e.g. 90%, 95%, 98% confidence level) over some defined period of time. For example, for a given temperature data set representing the body temperature of a user of some predefined time period (e.g. the last three days of temperature data for a user as provided by the workflow module), the heuristic module can be configured to average the body temperature measurements so as to identify an average body temperature over the period. As has been described, outlier values may have been filtered out at the preprocessor.

A measure of the variation in the temperature measurements may also be calculated so as to enable calculation of an upper bound of the average (e.g. baseline) temperature of the user over the predefined period to a predefined confidence level. For example, the upper bound of the average temperature may be identified to the 95% confidence level. FIG. 3 illustrates the upper 314 and lower 316 bounds at the 95% confidence level of a baseline temperature 315 calculated for the three days of temperature data shown in the figure.

The baseline temperature may be provided as a parameter to the neural network 111 for use as a bias value. This can help prevent saturation of the neurons of the neural network which may lead to out-of-bound predictions.

The upper bound on the baseline temperature can be used to identify temperature events in a time series—e.g. possible artefacts or fevers. Temperature events can be defined as occurring where a temperature time series crosses (i.e. increases through) the upper bound on the baseline temperature. Arrow 802 in FIG. 8 illustrates a high temperature event where the time series representing a mild fever event crosses though the upper bound 808 of the baseline temperature of the user at the 95% confidence level. To improve the identification of artefacts, it can be advantageous to determine one or more measures of the first derivate slope of the temperature time series (e.g. its thermal velocity) and/or the second derivative of the temperature time series (e.g. its thermal acceleration) over some predefined period with respect to a temperature event.

For example, a rising gradient 804 may be determined for the time series over a predefined period of time up to the start of the temperature event 802 (e.g. the point at which the time series first crosses the line). A suitable predefined period of time could be 30, 40, 50, 60, 70, 80 minutes prior to the start of the temperature event. The gradient may be the gradient of a straight line between the data point a predefined long period of time prior to the start of the temperature event and the data point closest to the start of the temperature event itself.

For example, a falling gradient 806 may be determined for the time series over a predefined period of time following the maximum 808 of a temperature event 802. Any suitable method for determining a peak value within the characteristic band sequence identified by the heuristic module may be used. A suitable predefined period of time could be 30, 40, 50, 60, 70, 80 minutes after the peak value. The gradient may be the gradient of a straight line between the data point closest to the peak of the temperature event and the data point the predefined period of time following the peak of the temperature event.

In some examples, a gradient may be determined for one or more of the bands (i.e. A, B, C, D etc.)—e.g. the gradient of a band may be an average gradient of the temperature data over the band.

Such gradient parameters can be further used to identify artefacts in a temperature time series since they provide information regarding trends in the temperature time series for a temperature event. Gradient parameters calculated for a temperature event may be readily associated with each characteristic band sequence identified by the heuristic module. For example, the gradient parameters of the closest temperature event to a given characteristic band sequence identified by the heuristic module may be associated with that characteristic band sequence.

The heuristic module is configured to apply a series of rules to a characteristic band sequence in order to determine whether the increase is indicative of, for example, an artefact or fever event. Suitable rules may be identified by examining temperature data sets captured from users which comprise known temperature events—e.g. fevers of various grades and/or types, showers, baths, saunas, exercise, etc.

For example, a mild fever as shown in FIG. 8 may be identified using the following rule, where S1 and S2 are, respectfully, rising and falling gradients:

$$If ( A > 10 \& B > 10 \& ( A + B ) > 60 \& ( B + C ) > 120 \& ( D + E + F ) < 30 \& S 1 < 2 \& S 2 > - 1 ) then “ Fever Event - Mild ”$$

Which can be expressed as:
If time in band A and band B is greater than 10 minutes each, and the total time in bands A and B is at least 60 minutes;
The total time in bands B and C is at least 120 minutes;
The total time in bands D, E and F is less than 30 minutes;
The rising slope S1 over the 60 minutes preceding the temperature event is less than 2 degrees per hour; and
The falling slope S2 over the 60 minutes following the temperature event is more than −1 degrees per hour.

Suitable rules can be identified to distinguish between each grade of fever as set out in table 1. In other examples, any method known in the art for algorithmically detecting the presence of a fever in a temperature time series may be used to identify fever events.

The heuristic module may be configured to identify a fever event in a temperature data set with reference to the time at which maximum of the fever event occurs. For example, a temperature time series may be provided by the heuristic module with a time reference and a grade of fever, which indicates where in the time series the fever event occurs and the determined grade of the fever. For a given identified fever event, the heuristic module may be configured to reference the onset of fever in a temperature data set by identifying the time at which the body temperature exceeds 38.1 degC. Thus, for example, the heuristic module may be configured to provide a temperature time series with a time reference to the onset of fever indicating where in the time series the onset of a fever event is considered to occur.

Note that references herein to temperature and body temperature are to core body temperature as measured or as estimated from the temperature measurements acquired from a user—for example, by wearable sensor 103.

Artefact events may alternatively or additionally be detected by the heuristic module according to the same multi-level threshold approach. FIG. 9 illustrates the impact on a temperature time series acquired by a skin worn wearable sensor of three showers taken by a user at different points in time. It will be appreciated that the variation in temperature exhibited during the showers is similar and characteristic. Each of the shower events (a)-(c) is depicted in the temperature time-series 902, 904 and 906. The time t₁ 908 spent in bands B and C for each of the showers is shown in the figure and can be observed to be similar for the three shower events. Typical shower events last approximately 15-30 minutes (users generally spend some time in the hot and humid bathroom environment following a shower) and predominantly present in bands B and C. Shower events can be identified, for example, using the following rule:

$$If ( B + C ) between 10 and 45 \& D < 10 \& S 1 > 1 \& S 2 \geq - 1 then “ Shower ”$$

Which can be expressed as:
If time in bands B and C is between 10 and 45 minutes;
The time in band D is less than 10 minutes;
The rising slope S1 over the 60 minutes preceding the temperature event is more than 1 degree per hour; and
The falling slope S2 over the 60 minutes following the temperature event is greater than-1 degree per hour.

Suitable rules can be empirically identified for other types of artefact (e.g. baths, saunas, exercise, faulty sensors) for a given location of the sensor on a user.

The heuristic module 107 may be configured to, on identifying an artefact event, discard the temperature values relating to the artefact and replace them with interpolated data points. The set of temperature values discarded may be all of those temperature values which lie in the set of defined bands used to characterise the artefact—e.g. for the shower artefact example above, all the data points in bands B, C and D. In some examples, the discarded temperature values of an artefact event may be all those which exceed the upper bound of the baseline temperature established for the time series being processed. This could be performed in the manner described above with reference to FIG. 3. In general, any suitable interpolation method may be used to generate data points to replace the discarded temperature values. Removing artefacts is advantageous because it can help to avoid the neural network identifying precursors in the features of artefact events and hence potentially resulting in a false prediction of fever or absence of fever.

The ovulation cycle in women can present challenges to the detection of fevers and precursors because shortly after ovulation (around 24 hours later) in the luteal phase, body temperature rises by around 0.3-0.7 degC for a period of around 11 days. For ovulating women it can be advantageous to correct for this variation in temperature. This can be achieved using information defining, for example, one or more of the point of ovulation, start of menstruation, and end of menstruation. Such information could be obtained in any suitable manner—for example, it could be entered by the user into the application 104 and provided to the data processing system. In some examples, such information could be provided by an automated system for monitoring the ovulation cycle, as is known in the art. The preprocessor and/or heuristic module may be configured to subtract a correction for a predefined number of days starting a predefined number of hours after the point of ovulation as determined according to any suitable algorithm based on the information provided by the user. For example, typically a correction of 0.5 degC for a period of 11 days starting 24 hours after the point of ovulation is appropriate, and these could be used as the default parameters of the correction. It can be advantageous to allow the user to specify the parameters of their cycle: e.g. the typical temperature change, length of the temperature change, and the time after their point of ovulation at which the temperature change starts. By using the values specific to a user, a more accurate correction can be achieved.

It will be appreciated that the ovulation cycle is well established and hence the point of ovulation may be straightforwardly estimated based on suitable information provided for an ovulation cycle. Information provided in respect of a previous cycle may be used to estimate the point of ovulation of a subsequent cycle. It can be advantageous to gradually (e.g. over a period of a few hours) increase the corrective subtraction from 0 to 0.5 degC at the start of the correction period so as to avoid introducing step changes into a temperature time series. Similarly, it can be advantageous to gradually (e.g. over a period of a few hours) decrease the corrective subtraction from 0 to 0.5 degC at the end of the correction period.

A similar approach may be used to define heuristics for identifying artefacts in movement data—e.g. patterns in movement data which are characteristic of, for example, exercise, travelling in a vehicle, showers, etc.

### Image Analysis

Following processing at the preprocessor and/or heuristic module, a temperature time series representing the body temperature and/or movement of a user is provided to a neural network for analysis in accordance with the principles described herein. The neural network is adapted to perform image classification. For example, in FIG. 1, the data processing system 102 includes a deep neural network 111 adapted to perform image classification.

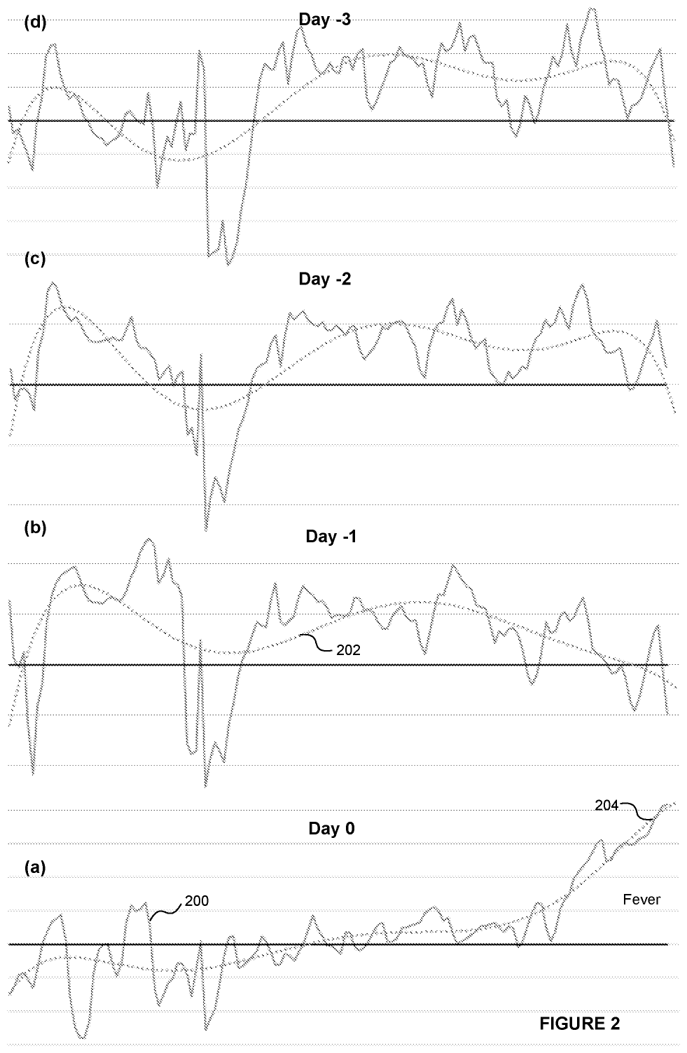

An exemplary time series 200 is shown in FIG. 2 which shows three days of temperature data prior to a fever event 204. The time series 200 may be the output of the heuristic module following preprocessing and the removal of artefacts. A smoothed signal line 202 has been added to illustrate the general changes in body temperature observed over the three day period. It is important to note however that it is the time series 200 (e.g. in the form of a scalogram) which is provided to the neural network, since this includes the higher frequency variations in which any precursors indicative of the approaching fever event will be present.

Sensor data, such as the temperature time series 200, is provided to the neural network in the form of images. The two-dimensional time series is processed so as to form a data set that comprises multi-dimensional data which can be represented as an image—this may be performed at, for example, the workflow module, or—as shown in FIG. 1—the transformation unit 108. The transformation unit 108 and/or preprocessor 106 may be configured to combine (e.g. average) one or more measurements so as to reduce the length of the time series. This can also help to smooth out noise in the data. For example, the transformation unit 108 may aggregate (e.g. by taking the mean) measurements so as to form a single value in respect of each 5 minute period. This results in 288 measurements every day.

In the case of movement data which is provided as vector information, the vector may be converted (e.g. at one or more of the sensor, preprocessor and transformation unit) into amplitude data representing the size of the movements, but not its direction. This is so as to provide scalar data for the scalogram.

The aggregated measurements (or simply the measurements received at the transformation unit if no aggregation is performed) are grouped together so as to form a time series of predefined length representing measurements (e.g. body temperature) of a user over a predefined period. For example, 3 days of measurements may be combined such that, for measurements aggregated over 5 minute periods, a time series is formed comprising 3*288=864 data points. The transformation unit is configured to process the time series so as to form a scalogram image representing that time series in a time-frequency space over the predefined period (in this example, 3 days). The scalogram includes amplitude, time and frequency domain information. Since the sequence is of a predefined length, the scalogram image is of predetermined pixel dimensions. Each data point in the sequence may correspond to a pixel in the scalogram. The transformation may be performed so as to generated a square scalogram (i.e. such that the number of pixels representing the frequency information is the same as the number of pixels representing the time domain-see FIG. 5). In some examples, the image may be resized using a suitable rescaling algorithm in order to match the topology of the input layer of the neural network. For instance, the image may be re-sized to 244×244 pixels which is often used by image classifier neural networks. In the present example, each scalogram transformation of a time-series is rescaled to form an image scalogram comprising 244×244 pixels.

In some examples, a separate scalogram is generated for each time series of sensor data—e.g. where both temperature and movement data is captured, a scalogram may be generated in the manner described herein for both the temperature data and the movement data. One or more neural networks 111 adapted to receive an input of the dimensions of the scalogram may be provided to process each scalogram. In some examples, the time series sensor data from multiple sensors may be combined into a single multi-dimensional scalogram—e.g. where both temperature and movement data is captured, a scalogram may be generated having four dimensions representing time, frequency, temperature and movement. Such a scalogram can be considered to be a four-dimensional image. Neural network 111 may be adapted to receive an input of the dimensions of the multi-dimensional scalogram so as to process the sensor data of each scalogram in parallel. It will be apparent to the skilled person that neural networks adapted to process inputs having a given number of dimensions can be readily modified to operate on inputs having a greater number of dimensions.

The transformation unit may be configured to transform the time series into a scalogram image using Continuous Wavelet Transform (CWT) and decomposition. This can have the advantage of increasing sensitivity to variation and high frequencies. A wavelet transform generates a three-dimensional matrix from a two-dimensional time series which can be visualised as a “scalogram” in which the pixels represent the values of the matrix in a time-frequency representation. Where more than one time-series is combined for representation in a single scalogram, the wavelet transform generates an N+2 dimensional matrix from the set of N two-dimensional time-series of sensor data. The matrix values (e.g. temperature data or an amplitude of a movement vector) may be depicted as colours and/or intensities according to a suitable scheme. An example of such a scalogram 500 is shown in FIG. 5 in which the time and frequency axes are indicated. The scalogram thus encodes features of the time series in a time-frequency representation. In other examples, the scalogram may be formed using a Fourier, Laplace, or wavelet transform of the time series.

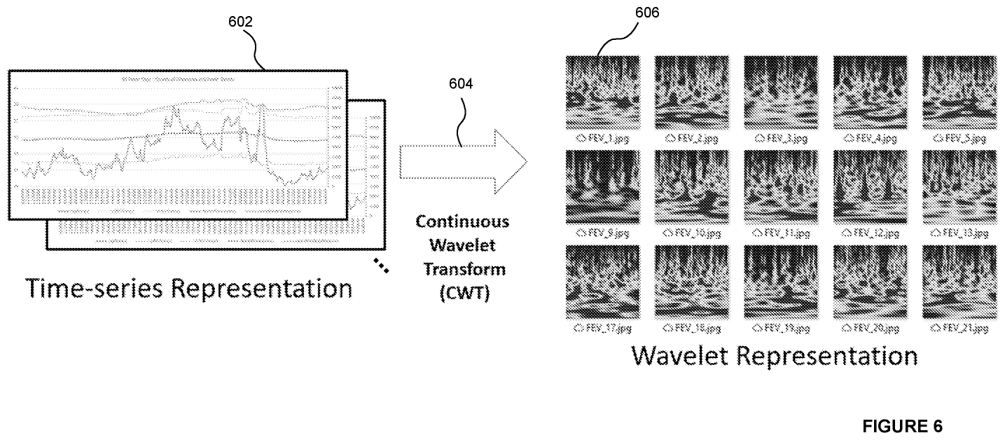

Typically the data processing system 102 will be configured to process sensor data from a plurality of different users—e.g. where the data processing system is a server arranged to receive sensor data from the sensors of a plurality of different users. For example, FIG. 6 illustrates the formation of a set of scalograms 606 from the temperature time series 602 of a plurality of users using Continuous Wavelet Transform (CWT). Each scalogram represents the variation on body temperature of the respective user over a predefined period of time.

A scalogram generated at the transformation unit is provided to the neural network—for example, deep neural network 111 in FIG. 1. Neural network 111 may be a deep neural network. Neural network 111 may include one or more convolutional layers. In general, the neural network may be any kind of network adapted to perform image or multi-dimensional data classification. That is, when provided with a scalogram the network is configured to detect the presence of one or more predefined patterns (e.g. image features in the time-frequency space) in the scalogram and to provide as its output an indication of that detection. Such networks are often used to tag images so as to automatically (i.e. without requiring human input) identify one or more objects in the image. For example, search engines use image classifier neural networks to index images available on the internet by keywords—e.g. images which include a cat will be associated with the keyword “cat”; images which contain a tree will be associated with the keyword “tree”; etc. Examples of image classifier neural networks include GoogLeNet, ResNet and AlexNet.

Neural network 111 is configured to perform image classification but, instead of identifying objects in visual images, the network is trained to identify features indicative of the presence of a fever precursor in scalograms representing sensor data in the time-frequency space. The presence of such precursors suggest that the user may develop a fever in the coming hours or days. The inventors have established that such precursors can be indicative of fever up to around three days in advance of the onset of fever. It is advantageous to configure the image classifier neural network 111 to identify precursor features of a fixed or substantially fixed size. For example, typically image classifier networks will look to identify a predefined pattern (e.g. a cat) of any scale in an image (i.e. a cat may occupy only a small portion of the image, or it may occupy substantially all the image).

The neural network may be trained in any suitable manner. For example, the neural network may be run on a set of training scalograms which include (a) scalograms representing a physiological parameter (e.g. body temperature) of a plurality of users over some predefined time period (e.g. 24, 36, 48, 60, 72 hours) prior to the onset of fever, and (b) scalograms representing that physiological parameter (e.g. body temperature) of a plurality of users over that same time period who did not go on to develop a fever. The neural network is adapted according to a suitable optimisation algorithm so as to identify a set of parameters (e.g. weights) for the network which best map its inputs (the scalograms) to its set of outputs—in this case a classifier indicating whether each scalogram does or does not include a precursor indicative of the onset of fever. The classifier may be binary (e.g. 0 or 1), numerical (e.g. a number in the range 0-1 such as the continuous output of a classifier network), or have any other suitable form. The output of the neural network may indicate a likelihood as to whether a scalogram does/does not contain one or more precursors predictive of the onset of fever. An exemplary approach to training the neural network is described below.

It is advantageous if the output of the neural network 111 is a continuous output (e.g. a number in the range 0-1) indicating the likelihood that a scalogram input to the network includes one or more precursors indicative of the onset of fever. Neural networks for performing image classification typically provide such a continuous output. A suitable threshold may be established above which the onset of fever is predicted (in the case that a higher value indicates a higher likelihood that the scalogram includes a precursor, otherwise values below the threshold may be predictive of the onset of fever). For example, a value above 0.6, 0.7, 0.8, or 0.9 may be predictive of the onset of fever. The continuous output value itself may be provided to the user (e.g. via the workflow module 109 in the manner described below) as an indication of the likelihood of the onset of fever. For example, a user may be provided with a prediction of the onset of fever when the continuous output exceeds 0.7, in which case the user is further provided with an estimate of the likelihood of fever as a percentage—e.g. in the examples described herein, if the continuous output were 0.83, the user would be given a likelihood of 83% that they may develop a fever within the next 3 days.

The neural network 111 may be an existing image classification network in which the classifier layer (typically the last layer of the network) has been replaced with a new classifier layer and re-trained with the new classifier layer so that the network indicates whether or not a scalogram includes a precursor or not (e.g. by providing a continuous output value). It can be advantageous to use a pre-trained neural network (e.g. GoogLeNet) with a re-trained output classifier. Such pre-trained neural networks have already been configured and adapted over millions of images to offer good image classification performance and can be readily adapted to perform classification of scalogram images by replacing one or more layers, including the final classifier layer. Neural networks adapted to identify fever precursors with very high performance (over 90% detection accuracy) can be achieved by re-training such image classifier networks using training sets of hundreds of scalograms (i.e. they can be smaller sets than the training sets typically used to train the source image classifier networks). A machine learning platform such as TensorFlow can be used to generate a suitable neural network.

In general, convolutional neural networks offer good image classification performance. For example, neural networks which include the following layers (from input to output) have demonstrated good performance: input layer>convolution layer>ReLu layer>maxpool layer>fully connected layer>softmax layer>classifier layer.

The workflow module is configured to provide scalograms representing the body temperature of a user over some predefined time period (e.g. 3 days) to the trained neural network 111. The predefined time period is preferably the same time period of the scalograms on which the neural network is trained—i.e. the scalogram images are of the same size. The output of the neural network is a classifier indicating whether or not each scalogram includes a precursor characteristic of the onset of fever (e.g. as a binary output and/or a continuous output).

The output of the neural network may be provided to the workflow module 109 for provision to the user 101 (e.g. via application 104) and/or clinical support 110 (e.g. a clinical team at a hospital at which the user is a patient). For example, on the neural network outputting a classifier indicative of the presence of a fever precursor in the sensor data of a particular user, the workflow module may be arranged to cause the application 104 (which may be running at the user's smartphone) to indicate to the user that they may suffer a fever within some predefined time period into the future. For example, on the neural network outputting a classifier indicative of the presence of a fever precursor in the sensor data of a particular user, the workflow module may be arranged to provide a suitable indication to clinical support 110 (e.g. by means of a message to a healthcare system used at a hospital) to indicate that the user (e.g. a patient at the hospital) may suffer a fever within some predefined time period into the future.

The predefined period of time into the future within which a fever may be expected may be the same predefined period of time represented by each scalogram provided to the neural network. This is because the neural network is trained on scalograms which include sensor data up to the onset of fever but not including the fever itself. Precursors identified in such scalograms are therefore typically indicative of the onset of fever at most after a period of time equal to the length of time represented by the scalogram. In other words, the earliest that a precursor could occur and be detected by the neural network is at the start of the predefined period represented by the scalogram, and since the neural network is trained on scalograms for which any fever event occurs immediately after the sensor data represented by the scalogram, then the longest amount of time between a precursor and the onset of fever is the length of the predefined period represented by the scalogram.

It has been established that neural networks configured in accordance with the principles described herein are able to detect fever precursors up to 3 days prior to the onset of fever itself. It is therefore advantageous if the predefined period is 3 days (or more). An output of such a neural network indicating the possible presence of a fever precursor may be considered to be indicative of the onset of fever within the next 3 days.

Typically the data processing system 102 will be configured to process sensor data from a plurality of different users. The sensor data (e.g. time series and/or scalogram) processed at the data processing system may be associated with each user in any suitable manner, e.g. by means of a unique identifier for each user which is stored with the sensor data. The workflow module may be configured to provide the output generated by the neural network in respect of a particular scalogram to a particular one of the users in dependence on the unique identifier associated with that scalogram.

### Real-Time Analysis

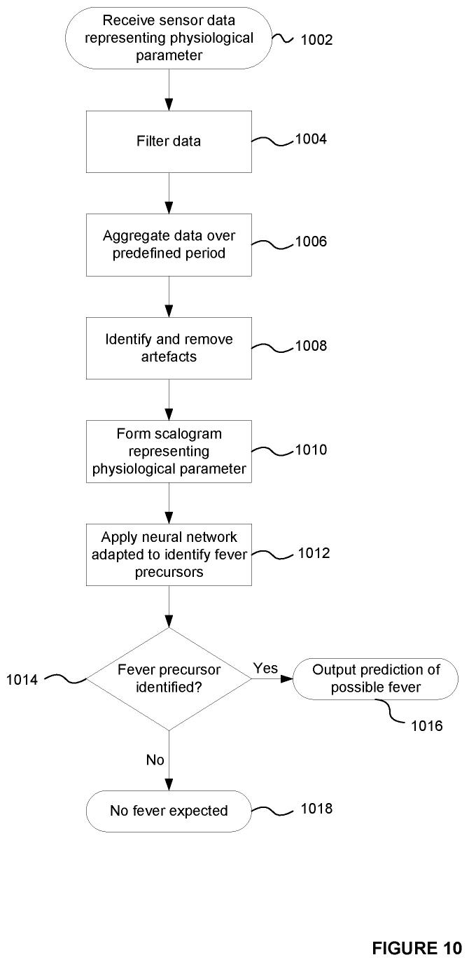

The analysis of sensor data acquired from users in accordance with the principles described herein will now be described in more detail by way of example with reference to FIG. 10. The sensor data may represent any suitable physiological parameter in which fever precursors may be identified (e.g. body temperature, movement, ECG, heart rate, heart rate variability, respiratory rate), with the same steps being performed as is described herein with respect to FIG. 10. Consider the exemplary system shown in FIG. 1 in which sensor data is acquired from a user by a body worn sensor 103. In this example, the sensor is configured to continuously measure a physiological parameter (e.g. temperature or movement) by sampling every 40 seconds and storing the samples. This generates a time series representing the physiological parameter of the user.

The body worn sensor periodically (e.g. every 30 minutes) transmits its stored sensor values by low energy Bluetooth to an application 104 running at the user's phone. The application periodically (e.g. every 30 minutes) transmits its stored sensor values to data processing system 102 which receives them 1002 for analysis. The temperature values may be subject to preprocessing (e.g. filtering 1004) at the preprocessor 106 in the manner described above.

Since the neural network 111 is configured to operate on scalograms of a predetermined size, the data processing system is configured to (e.g. at the workflow module and/or transformation unit) aggregate 1006 the sensor values until a sufficient number of samples are available to generate a scalogram. In the present example, the neural network is configured to operate on 3 days of sensor data and therefore 3 days of sensor samples are required to form a scalogram. Where less than 3 days of sensor data is available (e.g. the user has only recently started wearing the sensor) the data processing system (e.g. the workflow module and/or transformation unit) may pad out the sensor data with synthetic sensor values so as to provide 3 days of data. For example, suitable padding data may be used in place of the missing sensor values (e.g. zeros), an average of one or more of the earliest sensor values may be formed and that average value used as a constant sensor value for all the missing sensor values, or a predetermined constant sensor value could be used (e.g. in the case of temperature data, 37 or 37.5 degC). In this manner the neural network can be arranged to operate on fewer than 3 days of sensor data.

One or more of the heuristic module 107, transformation unit 108 and neural network may be configured to operate on the sensor data according to a predefined schedule and/or in response to receiving new sensor measurements. In some examples, the predefined schedule may be selectable by the user and or clinical support team (e.g. the user may choose between receiving indications of oncoming fever every hour, 3 hours, 6 hours, 12 hours, 24 hours). In the present example, the heuristic module, transformation unit and neural network are configured to operate on the received sensor data every three hours. In some examples, the heuristic module may be configured to operate on each new sensor data set as it is received at the data processing system and prior to formation of the sensor data set over the predefined period.

The workflow unit is configured to store the last three days of sensor data for the user. Periodically, according to a suitable schedule, or as each new set of sensor data is received, the workflow unit is configured to add the latest set of sensor data in sequence to the stored time series and discard an equivalent number of the oldest sensor values. The time series held by the workflow unit and representing the last 3 days of sensor data of the user is thus periodically updated with the latest available sensor values. In this manner, a sliding window of the last three days of sensor data is maintained by the workflow module.

In accordance with the predefined schedule (e.g. every three hours), the time series held by the workflow unit may be provided to the heuristic module 108 so as to filter out any artefacts (step 1008 in FIG. 10) and/or identify any fever events in the time series in the manner described above. It can be advantageous for the heuristic module to detect fever events in received data since some fevers can present without any precursors being evident in the preceding days. For example, fevers due to food poisoning or in response to medication can develop very quickly. The workflow module may be configured to alter the user via application 104 in response to the heuristic module detecting a fever.

In accordance with the predefined schedule (e.g. every three hours) the time series held by the workflow unit is provided to the transformation unit 108 so as to transform the time series into a scalogram 1010 in the manner described above. Thus, every six hours a new scalogram is formed which includes both the latest sensor data and some data overlapping with the time period represented by the preceding scalogram for the user. The new scalogram is provided to the neural network for analysis 1012 in the manner described above so as to detect the potential onset of fever through the identification of fever precursors in the scalogram. If fever precursors are detected at step 1014, a suitable output 1016 is provided to the user's application so as to alert the user to a potential fever in the next three days. Otherwise a suitable output 1018 is provided to the user's application indicating that no fever is expected in the next three days.

FIG. 4 illustrates the concept of identifying a fever 402 in four users A, B, C and D. Note that in these four cases a fever event 402 is observed. For each user, three days of data prior to the onset of fever are acquired by a body worn sensor and processed in accordance with the principles described herein. Various precursors indicative of the onset of fever may be observed in the sensor data variations of each user up to the onset of fever. A first precursor 404 is observed in the sensor data of users A and D. Precursor 404 is observed for user A almost three full days before the onset of fever and may therefore be detected by the data processing system described herein when the first scalogram including that precursor is analysed by the neural network around three days prior to the onset of fever.

Other precursors 406 and 408 are observed in the sensor data of the four users as shown in the Figure. Each of the precursors occurs at some point in the three days prior to the onset of fever as a characteristic feature of the sensor data acquired for the user. The sensor data (i.e. scalogram) of some users exhibits more than one precursor. The neural network need not be configured to distinguish between the different precursors—i.e. any precursor indicative of the onset of fever may be used to provide a binary indication as to whether or not the onset of fever is expected within the predefined period. The neural network may be configured to provide a continuous output representing a degree of match between features in the scalogram and fever precursors which the network has been trained to identify.

In some examples, the neural network may be trained to identify the underlying physiological cause (i.e. the aetiology) for the fever as well as predict the onset of fever. It has been established that fevers due to different physiological causes (aetiologies) present different characteristic precursors in a user's sensor data and can therefore be distinguished by the neural network. For example, viral and bacterial infections typically have different prodromal phases which can be distinguished by different or different combinations of precursors in the temperature time series of a user in the days prior to the onset of fever. Other causes of fever (e.g. in cystic fibrosis and COPD patients) can show other characteristic sets of precursors prior to the onset of fever. It is noted that some causes of fever (e.g. food poisoning) present so rapidly that precursors will not generally be observed.

The neural network can be arranged to identify the cause of a predicted fever by configuring the neural network to provide a multi-valued output that is predictive of both fever and its cause—i.e. by providing the neural network with a suitable classifier layer and suitably training the neural network. For example, the neural network may be adapted using a training data set according to a suitable optimisation algorithm so as to identify a set of parameters (e.g. weights) for the network which best map the inputs to the network (the scalograms) to a set of outputs which include: no predicted fever; predicted viral fever; predicted bacterial fever. Where other underlying causes of fever can be identified from precursors in the sensor data prior to the onset of fever, further output values can be provided associated with each additional underlying cause. Training of the neural network would be performed using a training data set that includes sensor data time series for a plurality of users exhibiting (a) no fever and (b) fever due to the range of underlying causes to be identified by the system, as is described in more detail below.

The neural network can be arranged to identify the grade or a range of grades of a predicted fever (e.g. according to table 1 above) by configuring the neural network to provide a multi-valued output that is predictive of both fever and its grade—i.e. by providing the neural network with a suitable classifier layer and suitably training the neural network. For example, the neural network may be adapted using a training data set according to a suitable optimisation algorithm so as to identify a set of parameters (e.g. weights) for the network which best map the inputs to the network (the scalograms) to a set of outputs which include: no predicted fever; predicted high fever; predicted moderate or mild fever. Training of the neural network would be performed using a training data set that includes sensor data time series for a plurality of users exhibiting (a) no fever and (b) fever of the range of grades to be identified by the system, as is described in more detail below.

As is known in the art, neural networks adapted to perform image classification can be configured to indicate where in an image the features are located which are associated with the particular output of the neural network. Neural networks configured in accordance with the principles described herein are adapted to detect the presence of precursors in scalograms generated from the sensor data of users. Since a scalogram represents the time series as a matrix of values with respect to frequency and time (see FIG. 5), the position of detected precursor features in the scalogram with respect to the time axis of the scalogram is indicative of the time at which the precursor feature occurred. For example, in FIG. 5 which is a scalogram representing three days of sensor data, the three consecutive days are indicated.

Consider circled feature 502 in the scalogram of FIG. 5. If this feature is a precursor indicative of the onset of fever then the neural network is likely to identify the feature and provide an output that the scalogram is predictive of the onset of fever, as described above.

In a neural network configured to provide the position in time of the feature 502 in the scalogram the neural network can further indicate the time at which the sensor precursor occurred. This information can be provided by the data processing system to the user to indicate when precursor events have been identified.

Data processing systems configured in accordance with the principles described herein operating on sensor data continuously acquired by wearable skin temperature sensors located on the chest under the armpit have been shown to predict fever in human users with a sensitivity of 94% and a specificity of 91%. The validation sensitivity (rate of true positives) is 93.0% and the validation specificity (rate of true negatives) is 87.7%. This results in a combined accuracy of 90.4%.

It has been identified that the likelihood of a detected precursor being predictive of the onset of fever depends on where the precursor is identified relative to the fever event. Precursors occurring the day before fever onset offer a prediction accuracy of 90.4%; precursors occurring two days before fever onset offer a prediction accuracy of 82.7%; precursors occurring three days before fever onset offer a prediction accuracy of 76.8%. It can be advantageous to provide these statistics to the user as a level of confidence in a prediction of the onset of fever. It can be advantageous to combine the continuous output from the neural network (which is indicative of the likelihood that a precursor has been identified in data provided to the network) with the prediction accuracy based on when the precursor is identified in the scalogram. For example, the two probabilities may be combined in accordance with Bayes Theorem so as to provide a conditional probability that a precursor is indicative of the onset of fever given the time at which the precursor has been identified in a scalogram. Note that this is an estimate since, when a given scalogram is analysed and a precursor identified in it, it is not apparent where the time period represented by that scalogram occurs with respect to the possible future fever event. For example, if the fever precursor is identified three days ago in a scalogram, then it is assumed that fever is imminent and the prediction accuracy is taken to be 76.8%; whereas if the fever precursor is identified one day ago in a scalogram, then fever (as predicted by a precursor at least) could be imminent or up to two days away—in this case the prediction accuracy may be taken to be 90.4% as an estimate when in fact in this case the fever could still be two days away with a prediction accuracy of 76.8%. Other approaches may be taken as appropriate to the information which it is desired to provide to the user/clinicians.

Predicting the onset of fever at an early stage up to 3 days before the fever itself enables prophylactic treatment such as antibiotics, antivirals and steroids to be administered in advance of the fever. This can reduce the severity of the fever or prevent the fever from occurring at all. Some fever events often start with a mild fever which can subsequently turn into a severe fever (e.g. due to a secondary infection). By treating the mild infection early, the sever fever may be prevented—as might hospitalization of the user. In users suffering from certain chronic conditions, such as COPD or cystic fibrosis, the early treatment of potential fevers can substantially minimize the severity of exacerbations.

In data processing systems which operate on multi-modal data (e.g. data from more than one type of sensor, such as temperature and movement sensors), a neural network 111 may be implemented in the manner described herein for independent operation on scalograms generated for the data of each modality. For example, a first neural network may be implemented for operation on scalograms representing body temperature data and a second neural network may be implemented for operation on scalograms representing movement data. Each neural network may be independently trained so as to perform classification for the specific modality on which it operates—for example, in the manner described below. The outputs of the independent neural networks may be combined using any suitable approach, for example, using mathematical algorithms such as thresholds, voting, or averaging, and/or using more complex methods such as genetic algorithms, fuzzy logic, shallow or deep neural networks. In general any suitable mathematical ensemble method for combining the outputs of the independent neural networks so as to optimize the combined output from the networks as a prediction of the onset of fever. As is known in the art, in cases where machine learning tools are used to combine the neural networks of each modality, an additional training step may be implemented to train the ensemble technology to optimize the output.

In some examples, the neural network can be adapted to identify fever events themselves, in addition to any precursors. This can be advantageous since it allows the data processing system to provide an indication as to when a user has had a fever (e.g. a short fever which might have otherwise been missed) and also enables rapid onset fevers which are often not preceded by precursors (e.g. due to food poisoning) to be identified as soon as possible. The neural network may be trained to detect fevers themselves by including in a training data set scalograms which comprise a fever event and applying the neural network to the training data set so as to adapt its parameters. The classifier layer of the neural network is configured to provide a ‘fever detected’ output in addition to outputs indicating that fever is/is not predicted. In this manner, when the neural network is adapted to the training data according to an optimisation algorithm, the neural network is trained to associate its possible set of outputs with input scalograms which include fever events, as well as input scalograms which include fever precursors. In some examples, a second neural network may be used to identify fever events in scalograms, with the first neural network being used to identify fever precursors in the manner described herein.

The neural network 111 may be any suitable implementation. For example, the neural network could be implemented in software and/or hardware at the data processing system. At least some of the neural network could be performed at a suitable hardware accelerator—e.g. a processor adapted to perform neural networks which include convolutional operations.

The methods and data processing systems described herein may be used to detect precursors of fevers which are the result of a range of different diseases, treatments and conditions. For example, the fever may be due to one or more of: viral and bacterial infections, immune reactions, asthma, cytokine storms, immunotherapy, immune suppression, transplant, COPD, sepsis, neutropenia, and CAR T-cell therapy.

### Training

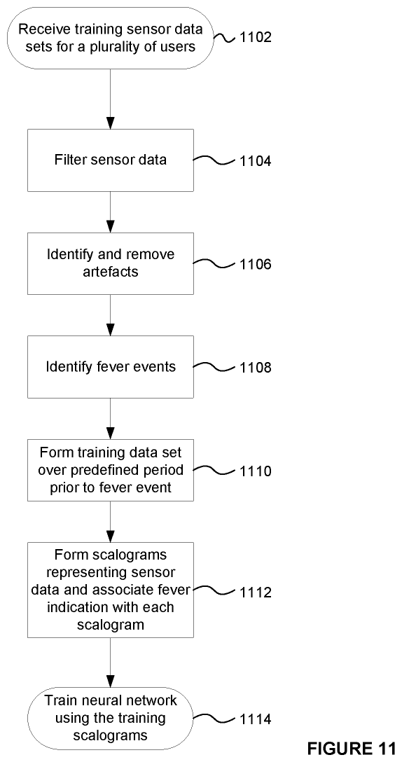

The training of a neural network to identify precursors in sensor data measurements predictive of the onset of fever in accordance with the principles described herein will now be described in more detail by way of example with reference to FIG. 11. The training of the neural network 111 may be performed at the data processing system 102. For example, the workflow module 109 may be configured to operate the neural network on training data sets according to an optimisation algorithm so as to adapt the neural network to identify precursors in scalograms and provide an output indicative of the possible onset of fever. In some examples, the neural network may be trained outside of the data processing system so as to identify a set of parameters (e.g. weights, biases, etc.). The trained neural network may then be implemented at the data processing system using those parameters. It shall be understood that references herein to training a neural network are to training any suitable implementation of the neural network, whether at the data processing system 102 or not.

The workflow module may be provided with a training mode in which it is configured to train the neural network 111 implemented at the data processing system in the manner described herein. The workflow module may additionally have a runtime mode in which it is configured to analyse data received from one or more users in accordance with the principles described herein—e.g. according to the method set out in FIG. 10.

In the particular example set out in the flowchart of FIG. 11, the neural network 111 at the data processing system is trained using data sets formed at the data processing system. At 1102, sensor data sets are received for a plurality of users. The data sets may be acquired in any suitable manner—e.g. using a wearable sensor arranged to continuously measure a physiological parameter (e.g. temperature) of a user. Each of the data sets includes at least one sensor data time series for a given user which is of a length at least of the predefined period of time over which the neural network is configured to operate—e.g. at least three days of data. Two approaches will now be described which can be used to provide suitable training data sets to the neural network.

In a first approach, the sensor data sets received at the data processing system are selected such that they include sensor data which exhibits fever events at known times, as well as sensor data which does not exhibit fever events. Such selection may be performed manually—e.g. by choosing data sets which represent the sensor data time series of users who are known to experience fever events in the periods represented in the data, as well as data sets which represent the sensor data time series of users who are known not to experience fever events in the periods represented in the data. For such data sets, step 1108 described below may be unnecessary.

In a second approach, the sensor data sets received at the data processing system include at least some sensor data time series for which it is not known whether the user experienced a fever or not. In this approach it can be advantageous to provide a large number (e.g. at least hundreds or thousands of temperature time series) so as to ensure that a sufficient number of fever events are identified to provide good training for the neural network. In this approach, the data processing system is arranged to identify fever events and the time at which they occur in received sensor time series data.

In some examples a hybrid approach may be used, where some of the sensor data sets are selected as representing known examples of fever events or no fever, and other sensor data sets are provided to the data processing system in order for the system to determine which data sets include fever and which do not.

In either approach, in the example shown in FIG. 11, the sensor data may be filtered 1104 at the preprocessor 106 and artefacts may be removed 1106 at the heuristic module 107 in the manner described above. The use of heuristics to remove artefacts which do not represent fever events can improve the performance of the neural network trained using data sets which have undergone artefact filtering, compared to those trained using data sets which have not undergone artefact filtering. This is because it can avoid the neural network associating the features of artefacts with fever events. It is advantageous that if artefacts are removed from the training data sets on which the neural network is trained, then artefacts are also removed from the data provided to the neural network at runtime for analysis.

For time series in the received data sets for which fever events have not already been identified (e.g. through manual selection of time series known to represent fever events), the data processing system 102 may be configured to identify fever events 1108 and the time at which they occur in the time series. For example, heuristic module 107 may be configured to perform fever detection in the manner described above. The heuristic module 107 may be configured to provide the time at which a fever event occurs by including as an output of the heuristic module the position in the time series (e.g. a time) at which an identified fever sequence starts, or the position of the associated event in the sensor data (e.g. in the case of body temperature data, this could be, for example, the point at which the temperature of the user increases through the upper bound of a measure of the user's average temperature at the 95% confidence level as the fever begins). This is discussed above with reference to the heuristic module 107.

In some examples, a neural network may be used to perform fever detection so as to identify time series which include a fever and the position in the time series at which the fever occurs. Such a neural network may be a second neural network (in addition to network 111) or may be neural network 111 trained to identify fever events (but potentially not yet fever precursors). The neural network may be trained to detect fevers in any suitable manner—e.g. for image classifier neural networks, by including in a training data set scalograms generated from time series in accordance with the principles herein which comprise a fever event and applying the neural network to the training data set so as to adapt its parameters. The neural network may be adapted to the training data according to an optimisation algorithm so that the neural network is trained to associate one or more outputs with input scalograms which include fever events. The neural network may be configured with a binary classifier layer to identify ‘fever’ or ‘no fever’ in the time series provided to it. The neural network is configured to provide the time at which an identified fever event occurs. For example, for an image classification neural network configured to operate on scalograms generated at a transformation unit as described herein, the neural network may be configured to provide the position in the scalogram at which the fever event is identified and hence the time at which the fever event occurs in the time series.

Step 1108 need not be performed for time-series for which it is already known whether the time-series includes a fever event—i.e. those that have already been classified, whether manually (e.g. by clinicians) or otherwise.

Whether or not the data processing system is configured to identify data sets which include fever events or otherwise, a training data set is formed 1110 which includes sensor data time-series associated with fevers and sensor data time-series which are not associated with fevers. Each time-series is in length equal to the predefined time period over which the neural network 111 is to operate—e.g. 3 days. Furthermore, each time-series which is associated with the onset of fever is selected from the training data received at the system so that it represents variations in a physiological parameter of the user over a period preceding the onset of fever and not including the fever itself. This may be achieved by, for each time-series which is identified (by any means) as including a fever event, selecting a predefined time period which ends a predetermined length of time prior to the position of the fever event in the time series. The position of the fever event could be the onset of the fever event—for example, the time of an event in the sensor data, or the start of a sequence of threshold bands in body temperature data indicative of fever, as identified by the heuristic module. The predetermined length of time prior to the position of the fever event in the time series may be a fixed time relative to the position of the fever event (e.g. 2, 4, 6, 8, 10, 12 hours prior to the position of the fever event), or may be the length of time to the start of the day on which the fever occurs such that the selected time series is the predefined period up to the start of the day on which fever occurs (e.g. where time series are to be 3 days long, the time series would include the preceding 3 days of sensor data up to the start of the day on which fever occurs). The formation of training data sets may be performed at the workflow module.

The training sets of sensor data time series are transformed 1112 by the transformation unit 108 into scalograms representing each time series in the manner described above with reference to the transformation unit 108. This results in a set of training scalograms, some of which represent variations in physiological parameters of users preceding the onset of a fever, and others which are not associated with a fever (i.e. do not represent fever or its onset within the predefined period of time). For each training scalogram, an indication is provided as to whether that scalogram is associated with a fever event (i.e. represents the variations in sensor data of a user preceding the onset of a fever).

At 1114 the workflow module is configured to train the neural network by applying the neural network to the training scalograms and, in dependence on an indication as to whether each scalogram is associated with a fever, adapt the parameters of the neural network (e.g. its weights) according to a suitable optimisation algorithm. In this manner the neural network may be trained to associate one or more outputs indicative of the onset of fever with input scalograms which include fever precursors but do not necessarily include fever events.

As has been mentioned above, the training approach described herein may be further extended to determine the severity and/or type of fever—e.g. the underlying cause of the fever, such as whether it is a viral or bacterial infection. This is achieved by provided each training scalogram associated with a fever with an indication as to the cause of the fever and/or the severity of the fever, and configuring the classifier layer of the neural network to offer a sufficient number of outputs to distinguish between the different causes of fever/severities of fever represented in the training data set. The severity of fever may be determined programmatically from a training data set which comprises time series including fevers of a range of severities. For example, the severity of a fever may be defined according to the core body temperature achieved by the patient which is available from sensor data representing body temperature. In order to train the neural network to distinguish between fevers having different underlying causes, the causes of fevers are required in the training data set and associated with the time series generated in accordance with the principles described above.

The data processing system of FIG. 1 is shown as comprising a number of functional blocks. This is schematic only and is not intended to define a strict division between different logic elements of such entities. Each functional block may be provided in any suitable manner. It is to be understood that intermediate values described herein as being formed by a data processing system need not be physically generated by the data processing system at any point and may merely represent logical values which conveniently describe the processing performed by the data processing system between its input and output.

Generally, any of the functions, methods, techniques or components described above can be implemented in software, firmware, hardware (e.g., fixed logic circuitry), or any combination thereof. The terms “module,” “functionality,” “component”, “element”, “unit”, “block” and “logic” may be used herein to generally represent software, firmware, hardware, or any combination thereof. In the case of a software implementation, the module, functionality, component, element, unit, block or logic represents program code that performs the specified tasks when executed on a processor. The algorithms and methods described herein could be performed by one or more processors executing code that causes the processor(s) to perform the algorithms/methods. Examples of a computer-readable storage medium include a random-access memory (RAM), read-only memory (ROM), an optical disc, flash memory, hard disk memory, and other memory devices that may use magnetic, optical, and other techniques to store instructions or other data and that can be accessed by a machine.

The terms computer program code and computer readable instructions as used herein refer to any kind of executable code for processors, including code expressed in a machine language, an interpreted language or a scripting language. Executable code includes binary code, machine code, bytecode, and code expressed in a programming language code such as C, Java or OpenCL. Executable code may be, for example, any kind of software, firmware, script, module or library which, when suitably executed, processed, interpreted, compiled, executed at a virtual machine or other software environment, cause a processor of the computer system at which the executable code is supported to perform the tasks specified by the code.

A processor, computer, or computer system may be any kind of device, machine or dedicated circuit, or collection or portion thereof, with processing capability such that it can execute instructions. A processor may be any kind of general purpose or dedicated processor, such as a CPU, GPU, System-on-chip, state machine, media processor, an application-specific integrated circuit (ASIC), a programmable logic array, a field-programmable gate array (FPGA), or the like. A computer or computer system may comprise one or more processors.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein. In view of the foregoing description it will be evident to a person skilled in the art that various modifications may be made within the scope of the invention.

1. A computer-implemented method of predicting fever comprising:
receiving a data sequence representing a physiological parameter of a user over a first period;
transforming the data sequence so as to form a scalogram representing the physiological parameter of the user over a second period;
analysing the scalogram at a neural network adapted to perform image classification so as to identify one or more fever precursors in the scalogram characteristic of the onset of fever; and
in response to identifying at least one of the one or more fever precursors in the scalogram, providing a prediction of the onset of fever in the user.

2. A method as claimed in claim 1, wherein the physiological parameter is body temperature and, optionally, one or more of movement, heart rate, respiration rate, and heart rate variability.

3. (canceled)

4. (canceled)

5. A method as claimed in claim 1, further comprising, prior to the transforming, discarding one or more of: physiological parameter measurements of the data sequence whose values lie above a predetermined upper bound and/or below a predetermined lower bound, physiological parameter measurements of the data sequence indicative of a rate of change of the physiological parameter over time which exceeds a predetermined rate, or physiological parameter measurements of the data sequence associated with one or more of a set of predefined artefacts.

6-10. (canceled)

11. A method as claimed in claim 1, wherein pixels of the scalogram represent values of the physiological parameter in the time-frequency domain.

12. A method as claimed in claim 1, wherein the first period is shorter than the second period.

13. A method as claimed in claim 12, further comprising, prior to the transforming, aggregating a plurality of data sequences representing the physiological parameter of the user over a plurality of first periods, the transforming being performed on the aggregate data sequence.

14. A method as claimed in claim 13, wherein the aggregating comprises, in an intermediate data set representing the physiological parameter of the user over the second period, appending to the intermediate data set as the most recent data the data sequence representing the physiological parameter of the user over the first period, and discarding a set of the oldest data from the intermediate data set equal in length to the first period.

15. A method as claimed in claim 1, further comprising calculating an average of the physiological parameter values of the user over the second period and, optionally, error bounds on that average at a predefined confidence level, wherein the average is provided to the neural network as a bias value for use when the neural network is analysing the scalogram.

16-18. (canceled)

19. A method as claimed in claim 1, wherein the providing a prediction of the onset of fever in the user is made in dependence on a continuous output of the neural network which is indicative of the likelihood of the at least one identified fever precursor being present in the scalogram.

20. A method as claimed in claim 19, wherein the at least one of the one or more fever precursors are identified in the scalogram if the continuous output of the neural network is above a predefined threshold.

21. (canceled)

22. A method as claimed in claim 1, wherein the neural network is adapted to perform image classification so as to identify the fever precursors in the scalogram and one or both of a measure of severity of the predicted fever and an indication of a cause of the predicted fever.

23. A method as claimed in claim 1, wherein the neural network is further adapted to perform image classification so as to identify a fever event in the scalogram.

24. (canceled)

25. A method as claimed in claim 1, wherein the neural network is configured to further provide a time at which a precursor is identified in the scalogram in dependence on the location of the precursor in the scalogram.

26. A method as claimed in claim 25, wherein the neural network is configured to, in dependence on the time at which a precursor is identified in the scalogram, provide an indication of the likely time period until the onset of fever in the user.

27. (canceled)

28. A method as claimed in claim 1, wherein the physiological parameter is movement and the received data sequence comprises a time series representing movement amplitude.

29. A method as claimed in claim 1, further comprising:
receiving a second data sequence representing a second physiological parameter of a user over the first period;
transforming the second data sequence so as to form a second scalogram representing the second physiological parameter of the user over the second period;
analysing the second scalogram at a second neural network adapted to perform image classification so as to identify one or more fever precursors in the second scalogram characteristic of the onset of fever; and
combining the outputs of the neural network and the second neural network so as to provide the prediction of the onset of fever in the user.

30. A method as claimed in claim 29, wherein the combining is performed at an ensemble layer of a third neural network adapted to combine the outputs of the neural network and the second neural network so as to provide the prediction of the onset of fever in the user.

31. A method as claimed in claim 1, wherein the received data sequence includes a plurality of physiological parameters, the transforming is performed so as to form a multi-dimensional scalogram representing the plurality of physiological parameters over the second period, and the neural network is adapted to perform image classification in the multi-dimensional scalogram.

32. (canceled)

33. A data processing system for predicting fever comprising:
an input unit configured to receive a data sequence representing a physiological parameter of a user over a first period;
a transformation unit configured to transform the data sequence so as to form a scalogram representing the physiological parameter of the user over a second period;
a neural network adapted to perform image classification and arranged to analyse the scalogram so as to identify one or more fever precursors in the scalogram characteristic of the onset of fever; and
an output unit configured to, in response to identifying at least one of the one or more fever precursors in the scalogram, provide a prediction of the onset of fever in the user.

34. (canceled)

35. A non-transitory computer readable storage medium having stored thereon computer readable instructions that, when executed at a computer system, cause the computer system to perform a computer-implemented method of predicting fever comprising:
receiving a data sequence representing a physiological parameter of a user over a first period;
transforming the data sequence so as to form a scalogram representing the physiological parameter of the user over a second period;
analysing the scalogram at a neural network adapted to perform image classification so as to identify one or more fever precursors in the scalogram characteristic of the onset of fever; and
in response to identifying at least one of the one or more fever precursors in the scalogram, providing a prediction of the onset of fever in the user.

36-53. (canceled)
